(19) United States
(12) Patent Application Publication
(10) Pub. No.: US 2018/0233220 A1
(43) Pub. Date: Aug. 16, 2018

(54) PHARMACOLOGICAL DISPENSING SYSTEM

(71) Applicant: Dayton Christian Trent, Amarillo, TX (US)

(72) Inventor: Dayton Christian Trent, Amarillo, TX (US)

(21) Appl. No.: 15/896,841

(22) Filed: Feb. 14, 2018

Related U.S. Application Data
(60) Provisional application No. 62/459,515, filed on Feb. 15, 2017.

(51) Int. Cl. G16H 20/13; G07F 17/00

(52) U.S. Cl. CPC G16H 20/13; G07F 7/005; G07F 17/0092

(57) ABSTRACT

A pharmacological dispensing system can consist of a medical drug prescription that is filled prior to the display of at least one clip of a multimedia package to an end user. The multimedia package can have safety information pertaining to the medical drug prescription. At least one clip of the multimedia package may be altered in response to a detected failure of the end user to understand the safety information. Confirmation of an understanding of the safety information by the end user after showing the end user the altered clip of the multimedia package prompts the dispensing of the medical drug prescription to the end user.

### RELATED APPLICATION

This application makes a claim of domestic priority under 35 U.S.C. 119(e) to U.S. Provisional Application No. 62/459,515 filed Feb. 15, 2017, the contents of which are hereby incorporated by reference.

### SUMMARY

A pharmacological dispensing system, in accordance with assorted embodiments, has a medical drug prescription that is filled prior to the display of at least one clip of a multimedia package to an end user. The multimedia package can have safety information pertaining to the medical drug prescription. At least one clip of the multimedia package may be altered in response to a detected failure of the end user to understand the safety information. Confirmation of an understanding of the safety information by the end user after showing the end user the altered clip of the multimedia package prompts the dispensing the medical drug prescription to the end user.

In other embodiments, a pharmacological dispensing system fills a medical drug prescription prior to generating a multimedia package that has a plurality of different clips with a controller. Each of the plurality of different clips has safety information pertaining to the medical drug prescription. A first clip of the plurality of different clips is customized to incorporate information from an end user's medical history and then displayed to an end user. A second clip of the multimedia package is altered prior to the controller confirming an understanding of the safety information of each of the plurality of different clips after showing the end user an entirety of the multimedia package. The confirmed end user understanding triggers the dispensing of the medical drug prescription to the end user.

A pharmacological dispensing system, in some embodiments, consists of a computing device having a controller, a sensor, and a screen with the screen configured to display a multimedia package pertaining to a medical drug prescription to an end user as directed by the controller. The controller is configured to alter at least one clip of the multimedia package in response to detection of a failure of the end user to understand safety information contained in the multimedia package with the sensor. The controller is then configured to release the medical drug prescription to the end user in response to confirmation of an understanding of the safety information after showing the end user the altered at least one clip.

### BRIEF DESCRIPTION OF THE DRAWINGS

FIG. 1 displays an example pharmacological dispensing system that may be employed in accordance with various embodiments.

FIG. 2 represents an example dispensing routine that may be employed by the pharmacological dispensing system of FIG. 1.

FIG. 3 depicts a block representation of an example pharmacological dispensing system arranged in accordance with some embodiments.

FIG. 4 conveys a block representation of a portion of an example pharmacological dispensing system operated in accordance with various embodiments.

FIG. 5 is an example multimedia adaptation routine that can be conducted with a pharmacological dispensing system in accordance with some embodiments.

FIG. 6 illustrates portions of an example pharmacological dispensing system that may be employed in accordance with various embodiments.

### DETAILED DESCRIPTION

The present disclosure generally relates to a pharmacological dispensing system that can optimize the efficiency, accuracy, and safety of the distribution of medication to an end user.

As computing systems and data distribution has evolved over time, medical prescriptions can be more quickly received by a medical pharmacy than if a prescription was to be manually delivered. Sophisticated pharmacy operations can increase the efficiency and accuracy of prescription filling. However, consulting with an end user of a filled prescription can be time consuming for a pharmacist and may not result in a proper level of understanding about the prescribed medication(s), prescribed dosage(s), or potential side effects. Hence, there is an opportunity for a pharmacological dispensing system to optimize prescription medication delivery to an end user that increases the end user's understanding of the medication while reducing the time a pharmacological professional spends engaging an end user.

Accordingly, various embodiments are directed to a pharmacological system that customizes a multimedia package to an end user to optimize the efficiency and understanding of a filled prescription. The ability to intelligently cater a multimedia package based at least on an end user's medical history, prescription history, and current real-time interaction increases the possibility that the end user will retain important information about the prescribed medication without occupying a pharmacological professional directly. The automated multimedia package of a pharmacological dispensing system can additionally reduce the liability of a medical pharmacy by ensuring the end user has a predetermined base level of understanding about the filled medication before relinquishing possession of the medication.

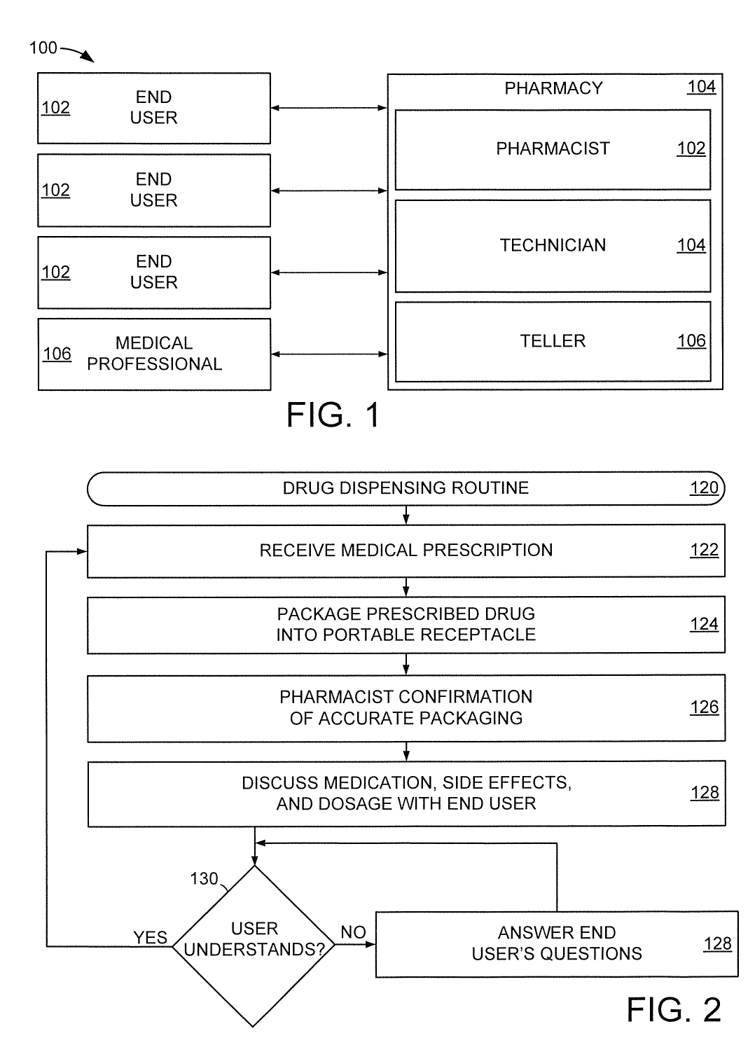

FIG. 1 displays a block representation of an example pharmacological dispensing system 100 in which assorted embodiments of this disclosure can be practiced. The dispensing system 100 can service any number of end users 102 that can engage one or more pharmacy locations 104 in a variety of different manners. For example, an end user 102 can engage a pharmacy 104 in person by being physically present at a retail location and/or digitally via a computing device, such as a computer, smartphone, tablet computer, or laptop computer.

The pharmacy 104 may also be engaged by medical professionals 106 that deliver medical prescriptions, or other information, that enables the pharmacy 104 to physically package one or more medical drugs into receptacle(s) that allow efficient dispensing by an end user 102. A pharmacy 104 can comprise any number and type of employee, but has at least one certified pharmacist 108 registered to fill and dispense medication in the state where the pharmacy 104 resides. One or more pharmacy technicians 110 may also be registered by a state and can be present in a pharmacy 104 to assist the pharmacist 108. Retail interactions in a pharmacy 104 may be facilitated by a teller 112 who is not registered to fill prescriptions, but can conduct point-of-sale operations.

The pharmacist 108, along with the technician 110 and teller 112, can engage in the example drug dispensing routine 120 shown in FIG. 2 to service a customer of the pharmacy 104. At any time and in any form, such as digital message, phone call, or personal delivery, one or more medical drug prescriptions are received by a pharmacy in step 122. The prescribed medical drug(s) are then packaged into at least one portable receptacle in step 124. Each packaged drug is confirmed by the pharmacist in step 126 to ensure accuracy of the packaging with the prescription, such as type of drug, number of pills, and external labeling. The confirmed packaging of a drug into a portable receptacle can be characterized as filling a prescription. However, filling a prescription does not extend to selling, or delivering, the portable receptacle to an end user.

The packaged drug(s) wait at the pharmacy, store, or assigned pick-up area until the person assigned the prescription, or an authorized representative of the prescribed person, arrives at the pharmacy to pick up and pay for the packaged drug(s). The person assigned the prescription can be characterized as an end user, which is also the only person authorized to consume the drug(s) of the filled prescription. A professional consultation between the end user, or their authorized representative, and a pharmacist is conducted in step 128 to discuss the prescription, such as the type, dosage, and side effects of the prescribed medications. The consultation of step 128 may further involve discussion of the end user's current symptoms in an effort to confirm the correct drug(s) were prescribed, although such confirmation is not required.

Decision 130 proceeds to determine if the end user, or authorized representative, understands the prescribed drug(s) and their usage. Confirmed understanding returns routine 120 to receiving new prescriptions in step 122 while a lack of understanding triggers step 132 to answer any and all questions the end user has about the prescribed drug(s). Although an important safety measure, it can be appreciated that the consultation between pharmacist and end user can be time consuming. Pharmacist consultations may additionally be complicated by the communication skills of the end user, such as diminished mental capacity, coherence, preoccupation with other mental activities, or verbal capabilities.

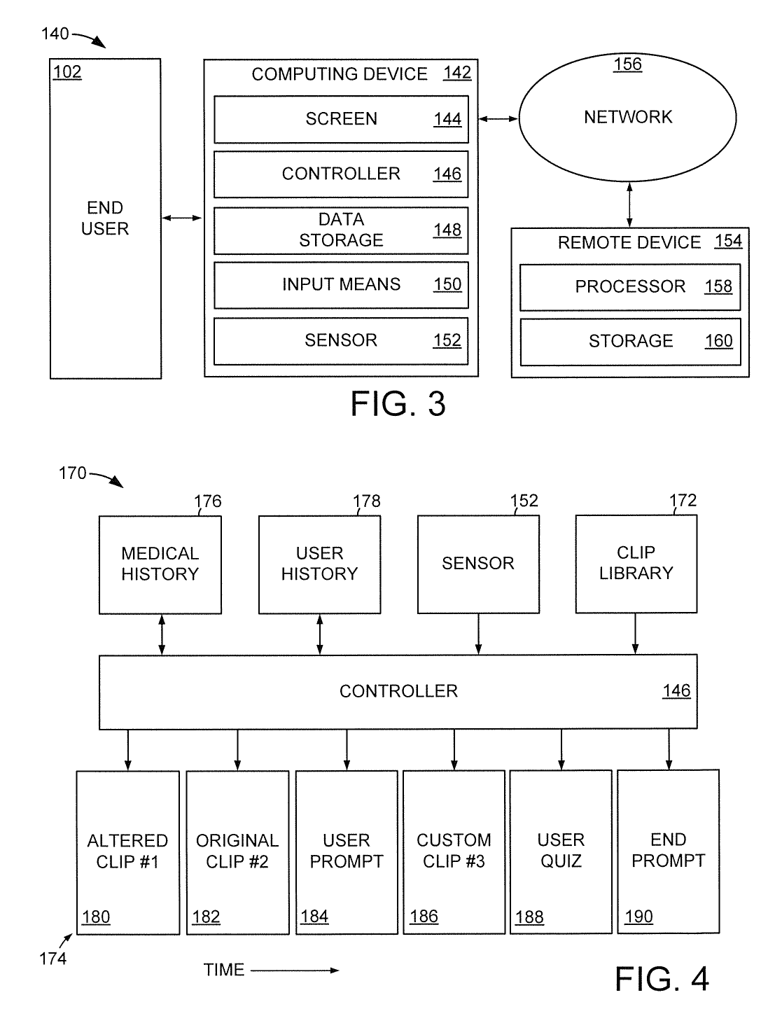

With these issues in mind, various embodiments are directed to increasing the efficiency, accuracy, and safety of filled prescription consultations by adding intelligence via at least one computing device to construct a multimedia consultation package customized to an end user based at least on the end user's medical history, which increases end user understanding of the prescribed drug(s), reduces the amount of time a pharmacist is occupied in consultations, and overcomes communication inefficiencies of an end user. FIG. 3 depicts a block representation of an example pharmacological dispensing system 140 configured to provide optimized pharmacological consultations in accordance with some embodiments.

The dispensing system 140 has at least one computing device 142 that engages one or more end users 102, or an end user's authorized representative, via one or more display screens 144. The screen 144 is not limited to a particular type and can be any visual display, such as virtual reality or augmented reality. It is contemplated that the screen 144 is complemented by an audio reproducing means, such as a speaker, headset, or phone, that allows auditory information to be conveyed to an end user 102. The computing device 142 may be present in a pharmacy or may be the property of the end user, such as a smartphone, tablet computer, or desktop computer, which allows the end user 102 to engage in pharmacological consultations at their convenience in locations other than a pharmacy.

The screen 144, and speaker, can convey information to the end user 102 as directed by a local controller 146, such as a microprocessor or other programmable circuitry. The controller 146 can utilize software, firmware, applications, and other data stored in local data storage 148, such as volatile and/or non-volatile solid-state memory and rotating magnetic memory, to generate and execute one or more multimedia packages via the screen/speaker 144. The computing device 142 can have one or more types of input means 150, such as a keyboard, mouse, biometrics, facial recognition, voice recognition, and gesture recognition, that allows the end user 102 to communicate questions and/or understanding of the drug information conveyed by the multimedia package. Such input means 150 may utilize one or more sensors 152 positioned in the computing device 142 to generate and/or execute the multimedia package and complete a pharmacological consultation with the same end result as routine 120.

While the computing device 142 may operate alone, various embodiments connect the computing device 142 to one or more remote devices 154 via a wired and/or wireless network 156. A remote device 154 can comprise any number of processors 158 and data storage 160 means that can operate in conjunction with the local computing device 142 to generate and execute a multimedia package that results in a pharmacological consultation. It is contemplated that the remote device 154 may be a pharmacy server that generates a multimedia package via a remote processor 158 that is stored first in the remote data storage 160 before being stored in the local storage 148 and executed on the screen/speaker 144 by the local controller 146.

FIG. 4 represents portions of an example pharmacological dispensing system 170 arranged in accordance with assorted embodiments. A local, or remote, controller 146 access a multimedia clip library 172 stored on local, or remote, data storage to construct a multimedia package 174 comprising at least one multimedia clips. It is noted that a multimedia clip can comprise video, audio, and haptic components that can be executed individually or concurrently by the computing device 142. The pharmacological dispensing system 170 can be utilized in any location separated from a pharmacy, such as a doctor's office, hospital, or end user's home. It is contemplated that the multimedia package be displayed on any screen, such as in a pharmacy, at a pharmacy drive-thru, or on a computing device of the end user.

In some embodiments, at least one clip of a multimedia package 174 will have drug interaction information. For instance, if the end user takes vitamins, make sure to take the vitamins 2 hours before or 2 hours after taking a prescribed antibiotic, such as ciprofloxacin. As another non-limiting example, a clip may advise avoiding consumption of grapefruit and/or drinking grapefruit juice with cholesterol medicine, such as simvastatin. One or more clips can educate end users on the proper use of medical dispensing apparatus, such as syringes, nebulizers, and epinephrine pens.

The clip library 172 may have one or more counseling points corresponding to a particular medical drug. Such counseling points may be stand-alone clips or additions to existing clips as directed by the controller 146. A counseling point may involve a recommendation that the end user take a laxative, such as Senokot or another pharmacist recommended laxative, based on their allergies, age, medical history, or current medication list, of the end user. Another non-limiting example of a counseling point prompts a pharmacist, technician, or teller to recommend an over-the-counter product, such as a probiotic, to complement the prescribed medical drug based on the end user's medical history. Counseling points may, in some embodiments, identify foods to avoid while taking a prescribed medical drug, foods to eat while taking a prescribed medical drug, drug storage instructions, indications of drug complications in the end user, and when to contact a medical professional while taking a prescribed medical drug.

The recommended product would be easily located in the store, Pharmacy or online.

The controller 146 can access and utilize the medical history 176 of a prescribed end user as well as a user history 178. It is contemplated that the medical history 176 and user history 178 can each be stored securely on a remote device located at a pharmacy and accessed by a controller 146 of a computing device via a secure connection, such as a password, encryption key, or certificate. The medical history 176 may comprise any number and type of medical information about a prescription drug end user, such as previous prescriptions, symptoms, allergies, time between prescriptions, and surgeries, that can contribute to the customization of one or more multimedia clips stored in the clip library 172.

The user history 178 may comprise any number and type of information about an end user, such as pick up preferences, insurance, previous drug questions, and multimedia clips watched. The information of the medical 176 and user 178 histories can collectively be utilized by a controller to select and customize clips from the clip library 172 to produce a multimedia clip 174 optimized for the end user to promote efficiency and accuracy of a pharmacological consultation. In the example clip 174 of FIG. 4, the controller 146 generates a first multimedia clip 180 followed by a different, second multimedia clip 182. The multimedia clips 180/182 may each correspond to a different medical drug, symptom, side effect, or dosage.

Either, or both multimedia clips 180/182 may be original, unaltered versions of clips stored in the clip library 172. Alternatively, the controller 146 may alter a multimedia clip stored in the clip library 172 based on the medical 176 and/or user 178 histories. For example, the controller 146 may alter the overall length of a clip, substitute portions of a clip with reserved content not in other clips, change language of the content of a clip, change the graphical delivery of a clip (i.e. animation to live action), or fill in open areas of a clip with content directly from the end user's histories 176/178. In another non-limiting example, the controller 146 may construct one or more questions from the various histories 176/178 that are interjected into a clip prior to a user prompt 184 section of the multimedia package 174.

A user prompt 184 is not limited to a particular type, length, or satisfaction criteria, but, in some embodiments, may comprise an active component, such as a question to be answered by the user, a polling of the user via an audible or visual response detected by a sensor 152, or a pause in video until the user triggers the next multimedia clip 186. The user prompt 184 may be passive in nature and may not require active engagement by the end user to trigger the next clip 186 to be played. For instance, a passive prompt may confirm eye contact of the end user with the screen or verify an attentive body language of the end user without pausing between, or during, playback of a multimedia clip 182/186.

As shown, the multimedia package 174 can comprise custom, original, and altered multimedia clips as designated by the controller 146. A custom clip is a clip that has been changed based on the end user's medical 176 and/or user 178 histories. An altered clip is a clip that has been changed based on the end user's user history 178 alone and an original clip is a clip that is unchanged from a clip stored in the clip library 172. The ability to customize individual multimedia clips as well as the placement of multiple clips in a package 174 along with active and/or passive prompts 184 allows the controller 146 to provide an optimized experience for the end user.

Despite an optimized experience, there is no guarantee that an end user will retain the pertinent information conveyed by the various multimedia clips. Hence, various embodiments conduct one or more quizzes 188 that confirm the end user's knowledge of selected prescription information, such as medicine name, pill identification, dosage, and side effects. A quiz 188 can comprise a written, visual, audible, and gesture questions that can be respectively be answered visually, audibly, haptically, or any combination thereof. The controller 146 may employ one or more sensors 152 to concurrently, or sequentially, register an end user's answer, such as a head nod, thumbs up, verbal confirmation, or button press.

A quiz 188 may be determined prior to the playing of the various multimedia clips or may be constructed by the controller 146 in response to logged user activity during the play of one or more multimedia clips. As a non-limiting example, the controller 146 may select different questions from a predefined question list based on the user's behavior (body language/verbal tone/attention) or may construct unique questions that verify an end user's understanding of the prescribed medication based on the user's behavior and/or answers to package prompt(s) 184. The ability to customize the manner in which an end user can verify an understanding of the information of the multimedia clips increases the chances that an end user will actively participate in the pharmacological consultation and return to the pharmacy for future prescription needs.

Once the controller 146 has confirmed the end user amply understands the pertinent information about the prescribed medication, the multimedia package 174 ends with a user end prompt 190 asking if the end user has any questions for a pharmacist. If so, the controller 146 can activate a real-time face-to-face interaction with a pharmacist in person, via phone, or via a video communication, which allows the end user to be in a pharmacy or separated from a pharmacy, such as at the end user's home. It is contemplated, but not required, that the multimedia package 174 can be activated by the end user at any time, but the end prompt 190 is only eligible to be answered when the end user is physically present at the pharmacy, which increases the chances the end user has any medication questions answered prior to receiving the prescribed medication.

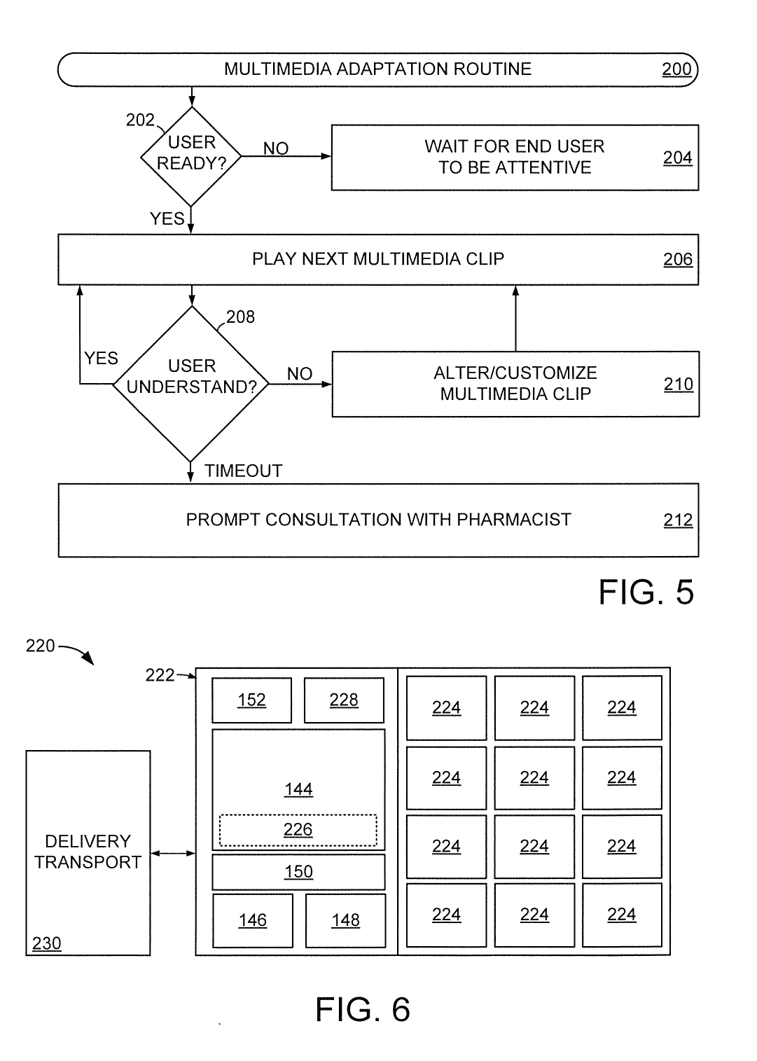

FIG. 5 is an example multimedia adaptation routine 200 that can be performed by the pharmacological dispensing system 170 of FIG. 4 in accordance with some embodiments. It is contemplated that the adaptation routine 200 is conducted by a controller that generates a multimedia package of one or more multimedia clips in response to the filled medicine. The multimedia package can be completely generated and queued, or can be partially generated prior to a controller polling at least one sensor in decision 202 to confirm an end user is ready to engage a multimedia package.

If the end user is not ready, such as not having a predetermined level of attentiveness, eye contact, and/or comfortable body language, step 204 waits to play a first multimedia clip. When decision 202 confirms the end user is ready, step 206 plays a multimedia clip selected from a locally stored clip library. The multimedia clip may be altered, or customized, in step 206, as directed by a controller, prior to being shown to an end user. As such, step 206 can comprise an evaluation of several different multimedia clips by a controller, selection of a clip predicted to result in efficient and thorough understanding of the prescribed medication, and customization of the length or content.

For instance, step 206 can insert facts and/or references to the user's medical history. In another non-limiting example, step 206 may involve a controller inserting previously recorded segment(s) based on past user activity, such as a segment indicating the medication matches the previous medication the user had filled or a segment adding more scientific facts in response to the user's attentiveness increasing after a scientific portion of a previous watched multimedia clip.

During and after multimedia clip playback, a controller can determine if the end user understands the content of the clip in decision 208. Such decision can be ascertained with passive prompts, active prompts, quiz results, past logged user history, and real-time detected user behavior. A lack of understanding triggers step 210 to change the previously played multimedia clip to increase the understanding of the clip's pertinent medical information. The clip modification of step 210 can involve altering, customizing, and/or moving a clip to a different location within the multimedia package. Once step 210 completes, or in the event the user understands the previously presented multimedia clip, step 206 is revisited to show the next multimedia clip to the end user. It is noted that the next clip in revisited step 206 may be a different version of a previously played clip, such as a clip containing content pertinent to a prescribed drug.

By cycling through step 206 and decision 208, an unlimited number of multimedia clips can be presented to an end user. The confirmed understanding, or at least user behavior indicative of understanding, increases the safety of medicine dispensing and decreases the liability of the pharmacy and pharmacist without involving the pharmacist directly in face-to-face consultations that can degrade the pharmacist's efficiency. However, a face-to-face consultation between pharmacist and end user can aid in the end user's understanding and comfort with the prescribed medicine. Hence, various embodiments execute routine 200 with the ability to request a face-to-face, real-time consultation with a pharmacist at any time either in person or digitally. A digital consultation may consist of streaming video and/or audio that is HIPAA certified to protect the end user.

A pharmacist consultation is prompted in step 212 in response to decision 208 failing to confirm an end user's understanding of one or more multimedia clips. Such failure, or additional questions from the end user, can be characterized as a timeout from the loop of step 206 and decision 208 that triggers step 212 to initiate a pharmacist consultation. It is contemplated that routine 200 ends with, or without, a face-to-face pharmacist consultation, such as in the event the end user understands all multimedia clips and has no additional questions for a pharmacist. At the conclusion of routine 200, the end user has a greater likelihood of understanding the prescribed medicine and enjoying the multimedia package due to the customization/alteration of clips and the real-time detection of inattentiveness and/or a lack of understanding.

A multimedia package can be utilized in variety of different pharmacological environments and is not limited strictly to retail pharmacies. FIG. 6 illustrates an example pharmacological dispensing system 220 that can employ a customized multimedia package to an end user in accordance with various embodiments. The dispensing system 220 comprises a self-contained housing 222 where a plurality of separate drug regions 224 are assigned by a local controller 146. Data resident in local storage 148 can complement the controller 146 in tracking that medications have been pharmacist approved and are properly delivered to an end user via an assigned drug region 224. The local data storage 148 can further temporarily, or permanently, store data pertaining to one or more end users, such as medical and/or user histories.

The storage of end user and medication data locally within the housing 222 can increase the efficiency of medication dispensing while providing heightened security compared to storage of data remotely. The computing components of the housing 222 can generate and convey a multimedia package to an end user via a display screen 144 and associated audio reproducing means (headset/speaker). An end user can communicate with the local controller 146 via any physical input means 150, such as a keyboard or touchscreen 226, and any passive input means detected by a primary 152 and/or secondary 228 sensor. For example, the primary sensor 152 may optically detect body language while the secondary sensor 228 audibly monitors tone of voice, inflection, and/or volume.

The ability to utilize different sensors 152/228 concurrently allow the controller 146 to more accurately identify an end user's attitude, behavior, and understanding of displayed multimedia clips pertaining to prescribed drugs resident in the respective drug regions 224 than if a single sensor was employed. Once the local controller 146 has sufficiently determined an end user has participated and understood a multimedia package about one or more filled medical drugs, the drug region 224 corresponding to the end user can be activated to dispense the drugs to the end user. As such, the housing 222 can provide an intelligent locker-type apparatus that educates the end user, confirms understanding of the prescribed medical drugs, and dispenses those medical drugs without human interference or inefficiencies.

It is contemplated that the local controller 146 can prompt human interaction either in person or digitally via the screen 144, upon prompting by an end user, system error, or a lack of understanding as a result of the multimedia package. While the housing 222 may be physically positioned anywhere electricity is available, various embodiments further employ one or more delivery transports 230 to physically take filled prescriptions to end users. A delivery transport 230 may be a human, human controlled robot, or automated robotic device, such as a wheeled or flying machine. The delivery transport 230 may also be utilized to bring filled prescriptions from a pharmacy to the housing 222 where the controller 146 directs movement of the prescription to an assigned drug region 224, such as via end effectors and/or conveyors.

In some embodiments, the delivery transport 230 travels directly to end users from a pharmacy instead of from a self-contained housing 222. The ability to selectively utilize any number, and type, of delivery transport increases the efficiency of prescription drug dispensing. However, it is noted that confirmation of an understanding of a multimedia package pertaining to prescribed medical drugs would be required prior to a delivery transport 230 bringing a prescription to an end user. For instance, an end user may participate in a multimedia package via a computing device where a controller 146 remote to the computing device employs at least one sensor of the computing device, such as a camera, microphone, positioning system, and other software, to ensure end user attentiveness and understanding of a customized/altered multimedia package in accordance with routine 200.

With a pharmacological dispensing system arranged and executed in accordance with assorted embodiments, greater numbers of filled prescriptions can be safely delivered to end users. By catering various multimedia clips to an end user while ensuring user understanding of the information of the clips increases the accuracy of medical drug usage without occupying a pharmacist's time for consultation on every filled prescription. In yet, the ability for an end user to request a face-to-face pharmacist consultation provides end user comfort and satisfaction when necessary.

The various embodiments of this disclosure serve as a tool to provide pharmacists and pharmacies needed time back to allow them to provide clinical services to patients such as Medication Therapy Management (MTM) services, immunizations, birth control prescribing and so forth. The use of multimedia clips as part of multimedia packages standardize pharmacist consultations or counseling to ensure patients receive correct prescription information 100% of the time. The questions incorporated into a pharmacological dispensing system allow for passive, and/or active, confirmation of an end user's understanding of important drug safety information, which can help us lower the $300 billion problem of nonadherence in the U.S., improve an end user's quality of life and significantly reduce healthcare costs, decreased hospital admissions, and lower numbers of unfavorable events as a result of increased drug understanding of how to take their prescriptions appropriately as prescribed.

It is to be understood that even though numerous characteristics and advantages of various embodiments of the present disclosure have been set forth in the foregoing description, together with details of the structure and function of various embodiments of the invention, this detailed description is illustrative only, and changes may be made in detail, especially in matters of structure and arrangements of parts within the principles of the present disclosure to the full extent indicated by the broad general meaning of the terms in which the appended claims are expressed.

What is claimed is:

1. A method comprising:
filling a medical drug prescription;
displaying at least one clip of a multimedia package to an end user, the multimedia package comprising safety information pertaining to the medical drug prescription;
altering at least one clip of the multimedia package in response to a detected failure of the end user to understand the safety information;
confirming an understanding of the safety information after showing the end user the altered at least one clip; and
dispensing the medical drug prescription to the end user.

2. The method of claim 1, wherein altering the at least one clip changes the length of the at least one clip.

3. The method of claim 1, wherein altering the at least one clip changes the content of the at least one clip.

4. The method of claim 3, wherein the content is changed in reference to a user history stored in local storage connected to the controller.

5. The method of claim 3, wherein the content is changed by introducing additional information about the medical drug prescription.

6. The method of claim 1, wherein a controller confirms understanding of the safety information by generating an active prompt for the end user.

7. The method of claim 1, wherein a controller confirms understanding of the safety information by generating a passive prompt for the end user.

8. The method of claim 1, wherein a controller confirms understanding of the safety information by generating a quiz for the end user.

9. The method of claim 1, wherein the medical drug prescription is dispensed without a face-to-face consultation by a pharmacist.

10. A method comprising:
filling a medical drug prescription;
generating a multimedia package comprising a plurality of different clips with a controller, each of the plurality of different clips comprising safety information pertaining to the medical drug prescription;
customizing a first clip of the plurality of different clips to incorporate information from an end user's medical history;
displaying the first clip of the plurality of different clips to an end user;
altering a second clip of the multimedia package;
confirming an understanding of the safety information of each of the plurality of different clips with the controller after showing the end user an entirety of the multimedia package; and
dispensing the medical drug prescription to the end user.

11. The method of claim 10, wherein the first clip is customized by referencing at least one prior medical drug prescription of the end user.

12. The method of claim 10, wherein the second clip is altered by the controller during the generating step and prior to being shown to the end user.

13. The method of claim 10, wherein the second clip is altered by the controller in response to a body language of the end user detected during the showing of the multimedia package by a sensor connected to the controller.

14. The method of claim 10, wherein the end user elects to see a pharmacist and the controller prompts a face-to-face consultation between the end user and the pharmacist prior to dispensing the medical drug prescription.

15. The method of claim 10, wherein the controller is resident in a computing device of the end user.

16. The method of claim 10, wherein the end user views the entirety of the multimedia package separated from a pharmacy.

17. The method of claim 10, wherein the controller displays at least one of the plurality of different clips to the end user multiple times prior to confirming the understanding of the safety information.

18. A system comprising a computing device comprising a controller, a sensor, and a screen, the screen configured to display a multimedia package pertaining to a medical drug prescription to an end user as directed by the controller, the controller configured to alter at least one clip of the multimedia package in response to detection of a failure of the end user to understand safety information contained in the multimedia package with the sensor, the controller configured to release the medical drug prescription to the end user in response to confirmation of an understanding of the safety information after showing the end user the altered at least one clip.

19. The system of claim 18, wherein the computing device is resident in a self-contained housing with a plurality of separate drug regions.

20. The system of claim 18, wherein the computing device is physically separated from any retail pharmacy location.
